(19) United States
(12) Patent Application Publication
(10) Pub. No.: US 2007/0189977 A1
(43) Pub. Date: Aug. 16, 2007

(54) Spray-on formulations and methods for dermal delivery of drugs

(76) Inventors: Jie Zhang, Salt Lake City, UT (US); Kevin S. Warner, West Jordan, UT (US); Sanja Sharma, Sandy, UT (US)

Correspondence Address: THORPE NORTH & WESTERN, LLP., 8180 SOUTH 700 EAST, SUITE 350, SANDY, UT, 84070, US

(21) Appl. No.: 11/640,132

(22) Filed: Dec. 14, 2006

Related U.S. Application Data
(63) Continuation-in-part of application No. 11/146,917, filed on Jun. 6, 2005.
(60) Provisional application No. 60/750,637, filed on Dec. 14, 2005.
(60) Provisional application No. 60/795,091, filed on Apr. 25, 2006.
(60) Provisional application No. 60/577,536, filed on Jun. 7, 2004.

(51) Int. Cl. A61K 9/12; A61K 31/573; A61K 31/57; A61K 31/275; A61K 31/19

(52) U.S. Cl. 424/45; 514/171; 514/569; 514/524

(57) ABSTRACT

The present invention is drawn to sprayable formulations, methods of drug delivery, and resultant solidified layers for dermal delivery of a drug. The formulation can include a drug, a non-volatile solvent system, a solidifying agent, and a propellant. The formulation can have an initial viscosity suitable to be expelled out of a pressurized or manual pump container and applied onto a skin surface as a layer. When applied to the skin, the formulation can form a solidified layer after at least a portion of the propellant is evaporated.

This application claims the benefit of U.S. Provisional Application No. 60/750,637, filed on Dec. 14, 2005, U.S. Provisional Application No. 60/795,091, filed on Apr. 25, 2006, and is a continuation-in-part of U.S. application Ser. No. 11/146,917, filed on Jun. 6, 2005, which claims the benefit of U.S. Provisional Application No. 60/577,536 filed on Jun. 7, 2004, each of which is incorporated herein by reference.

### FIELD OF THE INVENTION

The present invention relates generally to systems developed for dermal delivery of drugs. More particularly, the present invention relates to adhesive formulations having a viscosity suitable for sprayable application to a skin surface, and which form a sustained drug-delivery adhesive solidified layer on the skin.

### BACKGROUND OF THE INVENTION

In general, there are several kinds of transdermal or dermal drug delivery systems: skin patches, semisolids such as ointments creams and lotions, and spray-on formulations. Typical drug delivery patches are not elastic and have fixed shapes and sizes. They work best on skin areas that are relatively flat and that do not flex or stretch.

Typical semisolid dosage forms, such as ointments and creams, are often subject to unintended removal or transfer to other skin surfaces after being applied on the skin. The solvent in these semisolid formulations also tends to evaporate quickly after the application, which may negatively impact drug delivery rates. In addition, when a semisolid formulation is applied on skin, it is typically “rubbed in” which means only a very thin layer of the formulation is applied on the skin. This limits the amount of the drug that can be applied to each square centimeter of the skin, making sustained drug delivery difficult.

Spray-on formulations, such as those in pressurized containers or pumps, contain ingredients of traditional semisolid formulations plus propellants and/or diluents. The propellants and diluents improve the ease of application of the formulation. Therefore, besides the method of application, they have similar limitations and shortcoming as typical semisolids, as listed above.

In view of the shortcomings of many of the current dermal drug delivery systems, it would be desirable to provide systems, formulations, and/or methods that can i) provide sustained drug delivery over long periods of time; ii) are not vulnerable to unintentional removal by contact with clothing, other objects, or people for the duration of the application time; iii) can be applied to a skin area subject to stretching and expansion without causing discomfort or poor contact to skin; and/or iv) can be easily removed after application and use.

### SUMMARY OF THE INVENTION

The present invention relates to novel formulations that can be applied to a skin surface by spraying, and which can form a coherent, flexible, and/or continuous solid layer after the evaporation of the propellant in the formulation. Although film-forming technologies have been used in cosmetic and pharmaceutical preparations, typically, the solvents used in such systems do not last very long, and thus, are not optimal for sustained-release applications. In accordance with this, it has been recognized that the use of a non-volatile solvent system, specially selected or formulated for the selected drug and for the application needs, in the formulation can improve or even optimize sustained drug delivery. For example, the non-volatile solvent(s) in the formulations can be formulated or selected stay for the duration of the application of the drug and serve as vehicle solvent for the drug.

In accordance with this, a spray-on formulation for drug delivery can comprise a drug, a non-volatile solvent system comprising at least one non-volatile solvent, a solidifying agent, and a propellant. The formulation can have an initial viscosity suitable to be expelled out of a pressurized container or manual pump container and applied onto a skin surface as a layer, and further, the formulation can also be capable of forming a solidified layer on the skin surface after evaporation of at least a portion of the propellant.

In another embodiment, a method for dermal drug delivery can comprise spraying onto a skin surface an adhesive, solidifying formulation. The formulation can comprise a drug, a non-volatile solvent system that is flux-enabling for the drug, a solidifying agent, and a propellant. The formulation can have an initial viscosity suitable to be expelled out of a pressurized container and applied onto a skin surface as a layer. Additional steps include solidifying the formulation to form a solidified layer on the skin surface by at least partial evaporation of the propellant, and dermally delivering the drug from the solidified layer to the skin surface at a therapeutically effective rate over a sustained period of time.

Additional features and advantages of the invention will be apparent from the following detailed description which illustrate, by way of example, features of the invention.

### DETAILED DESCRIPTION OF THE PREFERRED EMBODIMENT(S)

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting, as the scope of the present invention will be defined only by the appended claims and equivalents thereof.

In describing and claiming the present invention, the following terminology will be used.

The singular forms “a,” “an,” and “the” include plural referents unless the context clearly dictates otherwise. Thus, for example, reference to “a drug” includes reference to one or more of such compositions.

“Skin” is defined to include human skin (intact, diseased, ulcerous, or broken), finger and toe nail surfaces, and mucosal surfaces that are usually at least partially exposed to air such as lips, genital and anal mucosa, and nasal and oral mucosa.

The term “drug(s)” refers to any bioactive agent that is applied to, into, or through the skin which is applied for achieving a therapeutic affect. This includes compositions that are traditionally identified as drugs, as well other bioactive agents that are not always considered to be “drugs” in the classic sense, e.g., peroxides, humectants, emollients, etc., but which can provide a therapeutic effect for certain conditions. When referring generally to a “drug,” it is understood that there are various forms of a given drug, and those various forms are expressly included. In accordance with this, various drug forms include polymorphs, salts, hydrates, solvates, and cocrystals. For some drugs, one physical form of a drug may possess better physical-chemical properties making it more amenable for getting to, into, or through the skin, and this particular form is defined as the “physical form favorable for dermal delivery.” For example the steady state flux of diclofenac sodium from flux enabling non-volatile solvents is much higher than the steady state flux of diclofenac acid from the same flux enabling non-volatile solvents. It is therefore desirable to evaluate the flux of the physical forms of a drug from non-volatile solvents to select a desirable physical form/non-volatile solvent combination.

The phrases “dermal drug delivery” or “dermal delivery of drug(s)” shall include both transdermal and topical drug delivery, and includes the delivery of drug(s) to, through, or into the skin. “Transdermal delivery” of drug can be targeted to skin tissues just under the skin, regional tissues or organs under the skin, systemic circulation, and/or the central nervous system.

The term “flux” such as in the context of “dermal flux” or “transdermal flux,” respectively, refers to the quantity of the drug permeated into or across skin per unit area per unit time. A typical unit of flux is microgram per square centimeter per hour. One way to measure flux is to place the formulation on a known skin area of a human volunteer and measure how much drug can permeate into or across skin within certain time constraints. Various methods (in vivo methods) might be used for the measurements as well. The method described in Example 1 or other similar method (in vitro methods) can also be used to measure flux. Although an in vitro method uses human epidermal membrane obtained from a cadaver, or freshly separated skin tissue from hairless mice rather than measure drug flux across the skin using human volunteers, it is generally accepted by those skilled in the art that results from a properly designed and executed in vitro test can be used to estimate or predict the results of an in vivo test with reasonable reliability. Therefore, “flux” values referenced herein can mean that measured by either in vivo or in vitro methods.

The term “flux-enabling” with respect to the non-volatile solvent system (or solidified layer including the same) refers to a non-volatile solvent system (including one or more non-volatile solvents) selected or formulated specifically to be able to provide therapeutically effective flux for a particular drug(s). For topically or regionally delivered drugs, a flux enabling non-volatile solvent system is defined as a non-volatile solvent system which, alone without the help of any other ingredients, is capable of delivering therapeutic sufficient levels of the drug across, onto or into the subject's skin when the non-volatile solvent system is saturated with the drug. For systemically targeted drugs, a flux enabling non-volatile solvent system is a non-volatile solvent system that can provide therapeutically sufficient daily doses over 24 hours when the non-volatile solvent system is saturated with the drug and is in full contact with the subject's skin with no more than 500 cm² contact area. Preferably, the contact area for the non-volatile solvent system is no more than 100 cm². Testing using this saturated drug-in-solvent state can be used to measure the maximum flux-generating ability of a non-volatile solvent system. To determine flux, the drug solvent mixture needs to be kept on the skin for a clinically sufficient amount of time. In reality, it may be difficult to keep a liquid solvent on the skin of a human volunteer for an extended period of time. Therefore, an alternative method to determine whether a solvent system is “flux-enabling” is to measure the in vitro drug permeation across the hairless mouse skin or human cadaver skin using the apparatus and method described in Example 1. This and similar methods are commonly used by those skilled in the art to evaluate permeability and feasibility of formulations. Alternatively, whether a non-volatile solvent system is flux-enabling can be tested on the skin of a live human subject with means to maintain the non-volatile solvent system with saturated drug on the skin, and such means may not be practical for a product. For example, the non-volatile solvent system with saturated drug can be soaked into an absorbent fabric material which is then applied on the skin and covered with a protective membrane. Such a system is not practical as a pharmaceutical product, but is appropriate for testing whether a non-volatile solvent system has the intrinsic ability to provide sufficient drug flux, or whether it is flux-enabling.

It is also noted that once the formulation forms a solidified layer, the solidified layer can also be “flux enabling” for the drug while some of the non-volatile solvents remain in the solidified layer, even after the volatile solvents (including water) have been substantially evaporated.

The phrase “effective amount,” “therapeutically effective amount,” “therapeutically effective rate(s),” or the like, as it relates to a drug, refers to sufficient amounts or delivery rates of a drug which achieves any appreciable level of therapeutic results in treating a condition for which the drug is being delivered. It is understood that “appreciable level of therapeutic results” may or may not meet any government agencies' efficacy standards for approving the commercialization of a product. It is understood that various biological factors may affect the ability of a substance to perform its intended task. Therefore, an “effective amount,” “therapeutically effective amount,” or “therapeutically effective rate(s)” may be dependent in some instances on such biological factors to some degree. However, for each drug, there is usually a consensus among those skilled in the art on the range of doses or fluxes that are sufficient in most subjects. Further, while the achievement of therapeutic effects may be measured by a physician or other qualified medical personnel using evaluations known in the art, it is recognized that individual variation and response to treatments may make the achievement of therapeutic effects a subjective decision. The determination of a therapeutically effective amount or delivery rate is well within the ordinary skill in the art of pharmaceutical sciences and medicine.

“Therapeutically effective flux” or “therapeutically sufficient flux” is defined as the permeation flux of the selected drug that delivers sufficient amount of drug into or across the skin to be clinically beneficial. “Clinically beneficial” or “clinically sufficient” when referring to flux means at some of the patient population can obtain some degree of benefit from the drug flux. It does not necessarily mean that most of the patient population can obtain some degree of benefit or the benefit is high enough to be deemed “effective” by relevant government agencies or the medical profession. More specifically, for drugs that target skin or regional tissues or organs close to the skin surface (such as joints, certain muscles, or tissues/organs that are at least partially within 5 cm of the skin surface), “therapeutically effective flux” refers to the drug flux that can deliver a sufficient amount of the drug into the target tissues within a clinically reasonable amount of time. For drugs that target the systemic circulation, “therapeutically effective flux” refers to drug flux that, via clinically reasonable skin contact area, can deliver sufficient amounts of the selected drug to generate clinically beneficial plasma or blood drug concentrations within a clinically reasonable time. Clinically reasonable skin contact area is defined as a size of skin application area that most subjects would accept. Typically, a skin contact area of 400 cm² or less is considered reasonable. Therefore, in order to deliver 4000 mcg of a drug to the systemic circulation via a 400 cm² skin contact area over 10 hours, the flux needs to be at least 4000 mcg/400 cm²/10 hour, which equals 1 mcg/cm²/hr. By this definition, different drugs have different “therapeutically effective flux.” Therapeutically sufficient flux” may be different in different subjects and or at different times for even the same subject. However, for each drug, there is usually a consensus among the skilled in the art on the range of doses or fluxes that are sufficient in most subjects at most times.

The following are estimates of flux for some drugs that are therapeutically effective or more than sufficient:

TABLE A
In vitro steady state flux values of various drugs

| | | Estimated Therapeutically |
|---|---|---|
| Drug | Indication | sufficient flux* (mcg/cm²/h) |
| Ropivacaine** | Neuropathic pain | 5 |
| Lidocaine | Neuropathic pain | 30 |
| Acyclovir | Herpes simplex virus | 3 |
| Ketoprofen | Musculoskeletal pain | 16 |
| Diclofenac | Musculoskeletal pain | 1 |
| Clobetasol | Dermatitis, psoriasis, | 0.05 |
| | eczema | |
| Betamethasone | Dermatitis, psoriasis, | 0.01 |
| | eczema | |
| Testosterone | Hypogonadal men, | 0.8 [ |
| Testosterone | Hormone treatment for | 0.25 |
| | postmenopausal women | |
| Imiquimod | Warts, basal cell | 0.92 |
| | carcinoma | |

*Flux determined using an in vitro method described in Example 1.
**Estimated flux based on known potency relative to lidocaine.

The therapeutically effective flux values in Table A (with the exception of ropivacaine) represent the steady state flux values of marketed products through hairless mouse or human epidermal membrane in an in vitro system described in Example 1. These values are meant only to be estimates and to provide a basis of comparison for formulation development and optimization. The therapeutically effective flux for a selected drug could be very different for different diseases to be treated for, different stages of diseases, and different individual subjects. It should be noted that the flux listed may be more than therapeutically effective.

The following examples listed in Table B illustrate screening of non-volatile solvent's flux enabling ability for some of the drugs specifically studied. Experiments were carried out as described in Example 1 below and the results are further discussed in the subsequent Examples 2-9.

TABLE B
In vitro steady state flux values of various
drugs from non-volatile solvent systems

| | | Average Flux* |
|---|---|---|
| Drug | Non-Volatile Solvent | (mcg/cm²/hr) |
| Betamethasone | Oleic acid | 0.009 ± 0.003 |
| Dipropionate | Sorbitan Monolaurate | 0.03 ± 0.02 |
| Clobetasol Propionate | Propylene Glycol (PG) | 0.0038 ± 0.0004 |
| | Light Mineral Oil | 0.031 ± 0.003 |
| | Isostearic acid (ISA) | 0.019 ± 0.003 |
| Ropivacaine | Glycerol | 1.2 ± 0.7 |
| | Mineral Oil | 8.9 ± 0.6 |
| Ketoprofen | Polyethylene glycol 400 | 5 ± 2 |
| | Span 20 | 15 ± 3 |
| Acyclovir | Polyethylene glycol 400 | 0 |
| | Isostearic acid + 10% | 2.7 ± 0.6 |
| | trolamine | |

*Each value represents the mean and st. dev of three determinations.

The in vitro steady state flux values in Table B from non-volatile solvents show surprising flux-enabling and non flux-enabling solvents. This information can be used to guide formulation development.

The term “plasticizing” in relation to flux-enabling non-volatile solvent(s) is defined as a flux-enabling non-volatile solvent that acts as a plasticizer for the solidifying agent. A “plasticizer” is an agent which is capable of increasing the percentage elongation of the formulation after the volatile solvent system has at least substantially evaporated. Plasticizers also have the capability to reduce the brittleness of solidified formulation by making it more flexible and/or elastic. For example, propylene glycol is a “flux-enabling, plasticizing non-volatile solvent” for the drug ketoprofen with polyvinyl alcohol as the selected solidifying agent. However, propylene glycol in a formulation of ketoprofen with Gantrez S-97 or Avalure UR 405 as solidifying agents does not provide the same plasticizing effect. The combination of propylene glycol and Gantrez S-97 or Avalure UR 405 is less compatible and results in less desirable formulation for topical applications. Therefore, whether a given non-volatile solvent is “plasticizing” depends on which solidifying agent(s) is selected.

Different drugs often have different matching flux-enabling non-volatile solvent systems which provide particularly good results. Examples of such are noted in Table C. Experiments were carried out as described in Example 1 below and the results are further discussed in the subsequent Examples 2-9.

TABLE C
In vitro steady state flux values of various drugs from particularly
high flux-enabling non-volatile solvent systems

| | High flux-enabling | Avg. Flux* |
|---|---|---|
| Drug | non-volatile solvent | (mcg/cm²/h) |
| Ropivacaine | ISA | 11 ± 2 |
| | Span 20 | 26 ± 8 |
| Ketoprofen | Propylene glycol (PG) | 90 ± 50 |
| Acycolvir | ISA + 30% trolamine | 7 ± 2 |
| Betamethasone | Propylene Glycol | 0.20 ± 0.07 |
| Dipropionate | | |
| Clobetasol | PG + ISA (Ratio of PG:ISA | 0.8 ± 0.2 |
| propionate | ranging from 200:1 to 1:1) | |

*Each value represents the mean and st. dev of three determinations.

It should be noted that “flux-enabling non-volatile solvent,” “flux-enabling, plasticizing non-volatile solvent,” or “high flux-enabling non-volatile solvent” can be a single chemical substance or a mixture of two or more chemical substances. For example, the steady state flux value for clobetasol propionate in Table C is a 9:1 for propylene glycol:isostearic acid mixture that generated much higher clobetasol flux than propylene glycol or ISA alone (see Table B). Therefore, the 9:1 propylene glycol:isostearic acid mixture is a “high flux-enabling non-volatile solvent” but propylene glycol or isostearic acid alone is not.

The term “adhesion” and “adhesive” when referring to a solidified layer herein refer to sufficient adhesion between the solidified layer and the skin so that the layer does not fall off the skin during intended use on most subjects. “Adhesive” or the like when used to describe the solidified layer can also mean the solidified layer is adhesive to the skin surface to which the initial formulation layer was originally applied (before the evaporation of the volatile solvent(s)). In one embodiment, it does not mean the solidified layer is adhesive on the opposing side. In addition, it should be noted that whether a solidified layer can adhere to a skin surface for the desired extended period of time partially depends on the condition of the skin surface. For example, excessively sweating or oily skin, or oily substances on the skin surface may make the solidified layer less adhesive to the skin. Therefore, the adhesive solidified layer of the current invention may not be able to maintain perfect contact with the skin surface and deliver the drug over a sustained period of time for every subject under any conditions on the skin surface. A standard is that it maintains good contact with most of the skin surface, e.g. 70% of the total area, over the specified period of time for most subjects under normal conditions of the skin surface and external environment.

The terms “flexible,” “elastic,” “elasticity,” or the like, as used herein refer to sufficient flexibility and elasticity of the solidified layer so that it is not broken or separate from the skin surface during the intended use. For example, a solidified layer that exhibits acceptably flexibility, elasticity, and adhesion to skin can be attached to human skin over a flexible skin location, e.g., elbow, finger, wrist, neck, lower back, lips, knee, etc., and will remain substantially intact on the skin upon stretching of the skin. It should be noted that the solidified layers of the present invention do not necessarily have to have any elasticity in some embodiments.

The term “peelable,” when used to describe the solidified layer, means the solidified layer can be lifted from the skin surface in one large piece or several large pieces, as opposed to many small pieces or crumbs.

The term “sustained” relates to therapeutically effective rates of dermal drug delivery for a continuous period of time of at least 30 minutes, and in some embodiments, periods of time of at least about 2 hours, 4 hours, 8 hours, 12 hours, 24 hours, or longer.

The use of the term “substantially” when referring to the evaporation of the volatile solvents means that a majority of the volatile solvents which were included in the initial formulation have evaporated. Similarly, when a solidified layer is said to be “substantially devoid” of volatile solvents, including water, the solidified layer has less than 10 wt %, and preferably less than 5 wt %, of the volatile solvents in the solidified layer as a whole.

The term “propellant” refers to a solvent which has a boiling point less than 20° C. and which can generate adequate pressure in a closed container at temperatures above 20° C. to expel the formulations of the present invention from the container. The propellant can be in dissolved in rest of the formulation, exist as a separate phase (separated or suspended) in the rest of the formulation, or exist in a separate enclosure (bag in can types) or compartment. When the propellant is maintained in a separate phase or compartment from the rest of the formulation, it can be mixed with the other components of the formulation prior to application to a skin surface, such as by shaking, inverting, agitation, or other manual or mechanical mixing methods.

“Volatile solvent system” refers to a single solvent or combination of solvents that are volatile, including water and solvents that are more volatile than water, but which have a boiling point which is greater than 25° C.

“Non-volatile solvent system” can be a single solvent or mixture of solvents that are less volatile than water. It can also contain substances that are solid or liquid at room temperatures, such as pH or ion-pairing agents. After evaporation of the volatile solvent system, most of the non-volatile solvent system should remain in the solidified layer for an amount of time sufficient to dermally delivery a given drug to, into, or through the skin of a subject at a sufficient flux for a period of time to provide a therapeutic effect. In some embodiments, in order to obtain desired permeability for an active drug and/or compatibility with solidifying agents or other ingredients of the formulation, a mixture of two or more non-volatile solvents can be used to form the non-volatile solvent system. In one embodiment, the combination of two or more non-volatile solvents to form a solvent system provides a higher transdermal flux for a drug than the flux provided for the drug by each of the non-volatile solvents individually. The non-volatile solvent system may also serve as a plasticizer of the solidified layer, so that the solidified layer is elastic and flexible.

“Adhesive solidifying formulation” or “solidifying formulation,” “sprayable solidifying formulation”, and the like, are used interchangeably and refer to a composition that has a viscosity suitable for application to a skin surface prior to evaporation of its volatile solvent(s), and which can become a solidified layer after evaporation of at least a portion of the volatile solvent(s). The solidified layer, once formed, can be very durable. In one embodiment, once solidified on a skin surface, the formulation can form a peel. The peel can be a soft, coherent solid that can be removed by peeling large pieces from the skin relative to the size of the applied formulation, and often, can be peeled from the skin as a single piece. The application viscosity is typically more viscous than a water-like liquid, but less viscous than a soft solid. Examples of preferred viscosities include materials that have consistencies similar to pastes, gels, ointments, and the like, e.g., viscous liquids that flow but are not subject to spilling. Thus, when a composition is said to have a viscosity “suitable for application” to a skin surface, this means the composition has a viscosity that is high enough so that the composition does not substantially run off the skin after being applied to skin, but also has a low enough viscosity so that it can be easily spread onto the skin. A viscosity range that meets this definition can be from about 100 cP to about 3,000,000 cP (centipoises), and more preferably from about 1,000 cP to about 1,000,000 cP.

In some embodiments of the present invention, it may be desirable to add an additional agent or substance to the formulation so as to provide enhanced or increased adhesive characteristics. The additional adhesive agent or substance can be an additional non-volatile solvent or an additional solidifying agent. Non-limiting examples of substances which might be used as additional adhesion enhancing agents include copolymers of methylvinyl ether and maleic anhydride (Gantrez polymers), polyethylene glycol and polyvinyl pyrrolidone, gelatin, low molecular weight polyisobutylene rubber, copolymer of acrylsan alkyl/octylacrylamido (Dermacryl 79), and various aliphatic resins and aromatic resins.

The terms “washable” or “removed by washing” when used with respect to the adhesive formulations of the present invention refers to the ability of the adhesive formulation to be removed by the application of a washing solvent using a normal or medium amount of washing force. The required force to remove the formulations by washing should not cause significant skin irritation or abrasion. Generally, gentle washing force accompanied by the application of an appropriate washing solvent is sufficient to remove the adhesive formulations disclosed herein. The solvents which can be used for removing by washing the formulations of the present invention are numerous, but preferably are chosen from commonly acceptable solvents including the volatile solvents listed herein. Preferred washing solvents do not significantly irritate human skin and are generally available to the average subject. Examples of washing solvents include but. are not limited to water, ethanol, methanol, isopropyl alcohol, acetone, ethyl acetate, propanol, or combinations thereof. In aspect of the invention the washing solvents can be selected from the group consisting of water, ethanol, isopropyl alcohol or combinations thereof. Surfactants can also be used in some embodiments.

The term “drying time” or “acceptable length of time” refer to the time it takes for the formulation to form a non-messy solidified surface after application on skin under standard skin and ambient conditions, and with standard testing procedure. It is noted that the word “drying time” in this application does not mean the time it takes to completely evaporate off the volatile solvent(s). Instead, it means the time it takes to form the non-messy solidified surface as described above.

“Standard skin” is defined as dry, healthy human skin with a surface temperature of between about 30° C. to about 36° C. Standard ambient conditions are defined by the temperature range of from 20° C. to 25° C. and a relative humidity range of from 20% to 80%. The term “standard skin” in no way limits the types of skin or skin conditions on which the formulations of the present invention can be used. The formulations of the present invention can be used to treat all types of “skin,” including undamaged (standard skin), diseased skin, or damaged skin. Although skin conditions having different characteristics can be treated using the formulations of the present invention, the use of the term “standard skin” is used merely as a standard to test the compositions of the varying embodiments of the present invention. As a practical matter, formulations that perform well (e.g., solidify, provide therapeutically effective flux, etc.) on standard skin can also perform well diseased or damaged skin.

The “standard testing procedure” or “standard testing condition” is as follows: To standard skin at standard ambient conditions is applied an approximately 0.1 mm layer of the adhesive solidifying formulation and the drying time is measured. The drying time is defined as the time it takes for the formulation to form a non-messy surface such that the formulation does not lose mass by adhesion to a piece of 100% cotton cloth pressed onto the formulation surface with a pressure of between about 5 and about 10 g/cm² for 5 seconds.

“Solidified layer” describes the solidified layer of an adhesive solidifying formulation after at least a portion of the volatile solvent system has evaporated. The solidified layer remains adhered to the skin, and is preferably capable of maintaining good contact with the subject's skin for substantially the entire duration of application under standard skin and ambient conditions. The solidified layer also preferably exhibits sufficient tensile strength so that it can be peeled off the skin at the end of the application in one piece or several large pieces (as opposed to a layer with weak tensile strength that breaks into many small pieces or crumbles when removed from the skin).

As used herein, a plurality of drugs, compounds, and/or solvents may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0 mm” should be interpreted to include not only the explicitly recited values of about 0.01 mm to about 2.0 mm, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. This same principle applies to ranges reciting only one numerical value. Furthermore, such an interpretation should apply regardless of the breadth of the range or the characteristics being described.

In accordance with this, a spray-on formulation for drug delivery can comprise a drug, a non-volatile solvent system comprising at least one non-volatile solvent, a solidifying agent, and a propellant. The formulation can have an initial viscosity suitable to be expelled out of a pressurized container or manual pump container and applied onto a skin surface as a layer, and further, the formulation can also be capable of forming a solidified layer on the skin surface after evaporation of at least a portion of the propellant.

In another embodiment, a method for dermal drug delivery can comprise spraying onto a skin surface an adhesive, solidifying formulation. The formulation can comprise a drug, a non-volatile solvent system that is flux-enabling for the drug, a solidifying agent, and a propellant. The formulation can have an initial viscosity suitable to be expelled out of a pressurized container and applied onto a skin surface as a layer. Additional steps include solidifying the formulation to form a solidified layer on the skin surface by at least partial evaporation of the propellant, and dermally delivering the drug from the solidified layer to the skin surface at a therapeutically effective rate over a sustained period of time.

Thus, these embodiments exemplify the present invention which is related to novel formulations, methods, and solidified layers. The formulations are sprayable onto skin surfaces, form solidified layers that can quickly (from 15 seconds to about 4 minutes under standard skin and ambient conditions) to moderately quickly (from about 4 to about 15 minutes under standard skin and ambient conditions) change into a solidified layer, e.g., a coherent and soft solid layer that is optionally peelable, for drug delivery. The solidified layer thus formed is capable of delivering drug to the skin, into the skin, or across the skin, etc., at a therapeutically effective rate over a sustained period of time, e.g., 30 minutes to tens of hours, so that most of the active drug that is delivered to the subject is delivered after the solidified layer is formed.

Additionally, the solidified layer formed by the formulations of the present invention typically adheres to the skin, but has a solidified, minimally-adhering, outer surface which is formed relatively soon after application and which does not substantially transfer to or otherwise soil clothing or other objects that a subject is wearing or that the solidified layer may inadvertently contact. The solidified layer can also be formulated such that it is highly flexible and stretchable, and thus capable of maintaining good contact with a skin surface, even if the skin is stretched during body movement, such as at a knee, finger, elbow, other joints, lips, etc.

To use the spray-on solidifying formulations of the present invention, the user sprays the formulation on the skin surface. The formulation forms a thin, wet layer on the skin surface. When the propellant (and the optional volatile solvent(s)) evaporates, the formulation solidifies into a thin, non-rigid, coherent, flexible, continuous, and/or preferably elastic solid layer. This solid layer has sufficient adhesion to the skin surface so that it can maintain good contact with the skin surface for the desired length of time, typically hours to tens of hours. Since the non-volatile solvent system does not evaporate and is designed to provide sufficient flux across the skin surface for the drug, the drug can be delivered from the solidified layer into or across the skin surface continuously for a sustained period of time or substantially for the entire duration of the application period.

In selecting the various components that can be used, e.g., drug, non-volatile solvent system, solidifying agent(s), propellant etc., various considerations may be applicable. For example, the propellant can be selected from pharmaceutically or cosmetically acceptable solvents known in the art to be useful for pressurized spray-on applications. Examples of propellants which can be used include hydrofluorocarbons, hydrochlorofluorocarbon, ethers or alkanes. More specifically, they include but are not limited to propane, butane, isobutane, pentane, isopentane, diethyl ether, dimethyl ether, 1,1 difluoroethane, 1,1,1,2 tetrafluorethane, 1,1,1,2,3,3,3-heptafluoropropane, 1,1,1,3,3,3 hexafluoropropane, vinyl chloride, compressed carbon dioxide, compressed nitrogen, or combinations thereof. The propellant can be in dissolved in the formulation, exist as a separate phase or as a suspended phase in the rest of the formulation, exist in a separate enclosure (bag in can types), or exist in a separate compartment and be mixed with the rest of the formulation to provide the propulsion at the application time. For most formulations, the weight percentage of the propellant can be from about 4 wt % to about 90 wt %, and more preferably from about 10 wt % to about 60 wt %.

Some embodiments of the present invention can also comprise a volatile solvent. Examples of volatile solvents which can be used include, but are not limited to, iso-amyl acetate, denatured alcohol, methanol, propanol, chlorobutanol, turpentine, cytopentasiloxane, cyclomethicone, methyl ethyl ketone, ethanol, isopropyl alcohol, water, ethyl acetate, acetone, or combinations thereof. When included in the formulations, these volatile solvents should be chosen to be compatible with the rest of the formulation. When used, it is desirable that an appropriate weight percentage of the volatile solvent(s) be present in the formulation. Too much of the volatile solvent system prolongs the drying time.

The non-volatile solvent system can also be chosen or formulated to be compatible with the solidifying agent, the drug, the propellant, and any other ingredients that may be present. For example, the solidifying agent can be chosen so that it is dispersible or soluble in the non-volatile solvent system. Most non-volatile solvent systems as a whole will be formulated appropriately after experimentation. For instance, certain drugs have good solubility in poly ethylene glycol (PEG) having a molecular weight of 400 (PEG 400, non-volatile solvent) but poor solubility in glycerol (non-volatile solvent) and water (volatile solvent). However, PEG 400 cannot effectively dissolve poly vinyl alcohol (PVA), and thus, is not very compatible alone with PVA, a solidifying agent. In order to dissolve sufficient amount of an active drug and use PVA as a solidifying agent at the same time, a non-solvent system including PEG 400 and glycerol (compatible with PVA) in an appropriate ratio can be formulated, achieving a compatibility compromise. As a further example of compatibility, non-volatile solvent/solidifying agent incompatibility is observed when Span 20 is formulated into a solidifying formulation containing PVA. With this combination, Span 20 can separate out of the formulation and form an oily layer on the surface of the solidified layer. Thus, appropriate solidifying agent/non-volatile solvent selections are desirable in developing a viable formulation and compatible combinations.

In further detail, non-volatile solvent(s) that can be used alone or in combination to form non-volatile solvent systems can be selected from a variety of pharmaceutically acceptable liquids. In one embodiment, the non-volatile solvent system can include glycerol, propylene glycol, isostearic acid, oleic acid, propylene glycol, trolamine, tromethamine, triacetin, sorbitan monolaurate, sorbitan monooleate, sorbitan monopalmitate, butanol, or combinations thereof. In another embodiment, the non-volatile solvent system can include benzoic acid, butyl alcohol, dibutyl sebecate, diglycerides, dipropylene glycol, eugenol, fatty acids such as coconut oil, fish oil, palm oil, grape seed oil, isopropyl myristate, mineral oil, oleyl alcohol, vitamin E, triglycerides, sorbitan fatty acid surfactants, triethyl citrate, or combinations thereof. In a further embodiment, the non-volatile solvent system can include 1,2,6-hexanetriol, alkyltriols, alkyldiols, acetyl monoglycerides, tocopherol, alkyl dioxolanes, p-propenylanisole, anise oil, apricot oil, dimethyl isosorbide, alkyl glucoside, benzyl alcohol, bees wax, benzyl benzoate, butylene glycol, caprylic/capric triglyceride, caramel, cassia oil, castor oil, cinnamaldehyde, cinnamon oil, clove oil, coconut oil, cocoa butter, cocoglycerides, coriander oil, corn oil, coriander oil, corn syrup, cottonseed oil, cresol, cyclomethicone, diacetin, diacetylated monoglycerides, diethanolamine, dietthylene glycol monoethyl ether, diglycerides, ethylene glycol, eucalyptus oil, fat, fatty alcohols, flavors, liquid sugars ginger extract, glycerin, high fructose corn syrup, hydrogenated castor oil, IP palmitate, lemon oil, lime oil, limonene, milk, monoacetin, monoglycerides, nutmeg oil, octyldodecanol, olive alcohol, orange oil, palm oil, peanut oil, PEG vegetable oil, peppermint oil, petrolatum, phenol, pine needle oil, polypropylene glycol, sesame oil, spearmint oil, soybean oil, vegetable oil, vegetable shortening, vinyl acetate, wax, 2-(2-(octadecyloxy)ethoxy)ethanol, benzyl benzoate, butylated hydroxyanisole, candelilla wax, carnauba wax, ceteareth-20, cetyl alcohol, polyglyceryl, dipolyhydroxy stearate, PEG-7 hydrogenated castor oil, diethyl phthalate, diethyl sebacate, dimethicone, dimethyl phthalate, PEG Fatty acid esters such as PEG-stearate, PEG-oleate, PEG-laurate, PEG fatty acid diesters such as PEG-dioleate, PEG-distearate, PEG-castor oil, glyceryl behenate, PEG glycerol fatty acid esters such as PEG glyceryl laurate, PEG glyceryl stearate, PEG glyceryl oleate, hexylene glycerol, lanolin, lauric diethanolamide, lauryl lactate, lauryl sulfate, medronic acid, methacrylic acid, multisterol extract, myristyl alcohol, neutral oil, PEG-octyl phenyl ether, PEG-alkyl ethers such as PEG-cetyl ether, PEG-stearyl ether, PEG- sorbitan fatty acid esters such as PEG-sorbitan diisosterate, PEG-sorbitan monostearate, propylene glycol fatty acid esters such as propylene glycol stearate, propylene glycol, caprylate/caprate, sodium pyrrolidone carboxylate, sorbitol, squalene, stear-o-wet, triglycerides, alkyl aryl polyether alcohols, polyoxyethylene derivatives of sorbitan-ethers, saturated polyglycolyzed C8-C10 glycerides, N-methyl pyrrolidone, honey, polyoxyethylated glycerides, dimethyl sulfoxide, azone and related compounds, dimethylformamide, N-methyl formamaide, fatty acid esters, fatty alcohol ethers, alkyl-amides (N,N-dimethylalkylamides), N-methyl pyrrolidone related compounds, ethyl oleate, polyglycerized fatty acids, glycerol monooleate, glyceryl monomyristate, glycerol esters of fatty acids, silk amino acids, PPG-3 benzyl ether myristate, Di-PPG2 myreth 10-adipate, honeyquat, sodium pyroglutamic acid, abyssinica oil, dimethicone, macadamia nut oil, limnanthes alba seed oil, cetearyl alcohol, PEG-50 shea butter, shea butter, aloe vera juice, phenyl trimethicone, hydrolyzed wheat protein, or combinations thereof. In yet a further embodiment, the non-volatile solvent system can include a combination or mixture of non-volatile solvents set forth in the any of the above discussed embodiments.

In addition to these and other considerations, the non-volatile solvent system can also serve as plasticizer in the adhesive formulation so that when the solidified layer is formed, the layer is flexible, stretchable, and/or otherwise “skin friendly.”

Certain ingredients in the formulations may be irritating to the skin may be desirable to use to achieve the desired solubility and/or permeability of the drug. In those cases, it is desirable to add compounds that are both capable of preventing or reducing skin irritation and are compatible with the formulation. For example, in a formulation where the propellant is irritating to the skin, it would be helpful to use a non-volatile solvent that is capable of reducing skin irritation. Examples of solvents that are known to be capable of preventing or reducing skin irritation include, but are not limited to, glycerin, honey, and/or propylene glycol.

The formulations of the present invention may also contain two or more non-volatile solvents that independently are not flux-enabling non-volatile solvents for a drug, but when formulated together become flux enabling non-volatile solvents as part of a system. One possible reason that these initially non-flux-enabling non-volatile solvents become flux-enabling non-volatile solvents when formulated together may be due to the optimization of the ionization state of the drug to a physical form which has higher flux, or alternatively, the non-volatile solvents together act in some other synergistic manner. One further benefit of the mixing of the non-volatile solvents is that it may optimize the pH of the formulation or the skin tissues under the formulation layer to minimize irritation. Examples of suitable combinations of non-volatile solvents that result in adequate non-volatile solvent systems for certain drugs include, but are not limited to, isostearic acid/trolamine, isostearic acid/diisopropyl amine, oleic acid/trolamine, propylene glycol/isostearic acid.

The selection of the solidifying agent(s) can also be carried out in consideration of the other components present in the adhesive formulation. The solidifying agent can be selected or formulated to be compatible to the drug and the propellants and the non-volatile solvent system (and optionally, other volatile solvents that are not propellants), as well as to provide desired physical properties to the solidified layer once it is formed. Depending on the drug, solvent, and/or other components that may be present, the solidifying agent can be selected from a variety of agents. In one embodiment, the solidifying agent can include polyvinyl alcohol with a MW range of 20,000-70,000 (Amresco), esters of polyvinylmethylether/maleic anhydride copolymer (ISP Gantrez ES-425 and Gantrez ES-225) with a MW range of 80,000-160,000, neutral copolymer of butyl methacrylate and methyl methacrylate (Degussa Plastoid B) with a MW range of 120,000-180,000, dimethylaminoethyl methacrylate-butyl methacrylate-methyl methacrylate copolymer (Degussa Eudragit E100) with a MW range of 100,000-200,000, ethyl acrylate-methyl methacrylate-trimethylammonioethyl methacrylate chloride copolymer with a MW greater than 5,000 or similar MW to Eudragit RLPO (Degussa), Zein (prolamine) with a MW greater than 5,000 such as Zein with a MW around 35,000 (Freeman industries), pregelatinized starch having a MW similar to Instant Pure-Cote B793 (Grain Processing Corporation), ethyl cellulose MW greater than 5,000 or MW similar to Aqualon EC N7, N10, N14, N22, N50, or N100 (Hercules), fish gelatin having a MW 20,000-250,000 (Norland Products), gelatin, other animal sources with MW greater than 5,000, acrylates/octylacrylamide copolymer MW greater than 5,000 or MW similar to National Starch, and Chemical Dermacryl 79.

In another embodiment, the solidifying agent can include ethyl cellulose, hydroxy ethyl cellulose, hydroxy methyl cellulose, hydroxy propyl cellulose, hydroxypropyl methyl cellulose, carboxymethyl cellulose, methyl cellulose, polyether amides, corn starch, pregelatinized corn starch, polyether amides, shellac, polyvinyl pyrrolidone, polyisobutylene rubber, polyvinyl acetate phthalate or combinations thereof. In a further embodiment the solidifying agent can include ammonia methacrylate, carrageenan, cellulose acetate phthalate aqueous such as CAPNF from Eastman, carboxy polymethylene, cellulose acetate (microcrystalline), cellulose polymers, divinyl benzene styrene, ethylene vinyl acetate, silicone, guar gum, guar rosin, gluten, casein, calcium caseinate, ammonium caseinate, sodium caseinate, potassium caseinate, methyl acrylate, microcrystalline wax, polyvinyl acetate, PVP ethyl cellulose, acrylate, PEG/PVP, xantham gum, trimethyl siloxysilicate, maleic acid/anhydride colymers, polacrilin, poloxamer, polyethylene oxide, poly glactic acid/poly-l-lactic acid, turpene resin, locust bean gum, acrylic copolymers, polyurethane dispersions, dextrin, polyvinyl alcohol-polyethylene glycol co-polymers, methyacrylic acid-ethyl acrylate copolymers such as BASF's Kollicoat polymers, methacrylic acid and methacrylate based polymers such as poly(methacrylic acid), or combinations thereof. In another embodiment, the solidifying agent can include a combination of solidifying agents set forth in the any of the above discussed embodiments. Other polymers may also be suitable as the solidifying agent, depending on the solvent(s), the drug, and the specific functional requirements of the given formulation. Other polymers may also be suitable as the solidifying agent, depending on the solvent(s), the drug, and the specific functional requirements of the given formulation.

In one embodiment, the non-volatile solvent system and the solidifying agent(s) should be compatible with each other. Compatibility can be defined as i) the solidifying agent does not substantially negatively influence the function of the non-volatile solvent system, except for some reduction of flux; ii) the solidifying agent can hold the non-volatile solvent system in the solidified layer so that substantially no non-volatile solvent oozes out of the layer, and/or iii) the solidified layer formed with the selected non-volatile solvent system and the solidifying agent has acceptable flexibility, rigidity, tensile strength, elasticity, and adhesiveness to skin. The weight ratio of the non-volatile solvent system to the solidifying agent(s) can be from about 0.1:1 to about 10:1. In another aspect, the weight ratio of the non-volatile solvent system to the solidifying agent can be from about 0.2:1 to about 4:1, and more preferably from about 0.5:1 to about 2:1.

The flexibility and stretchability of a solidified layer, which is optionally also a peel, can be desirable in some applications. For instance, certain non-steroidal anti-inflammatory agents (NSAIDs) can be sprayed directly over joints and muscles to form a solidified layer for transdermal delivery into joints and muscles. However, skin areas over joints and certain muscle groups are often significantly stretched during body movements. Such movement prevents non-stretchable patches from maintaining good skin contact. Lotions, ointments, creams, gels, foams, pastes, or the like also may not be suitable for use for the reasons cited above. As such, in transdermal delivery of NSAIDs into joints and/or muscles, the sprayable solidifying formulations of the present invention can offer unique advantages and benefits. It should be pointed out that although good stretchability can be desirable in some applications, the sprayable solidifying formulations of the present invention do not always need to be stretchable, as certain applications of the present invention do not necessarily benefit from this property.

A further feature of a formulation prepared in accordance with embodiments of the present invention is related to drying time. If the formulation dries too slowly, the subject may have to wait a long time before resuming normal activities (e.g. putting clothing on) that may remove un-solidified formulation. Thus, it is desirable for the drying time to be shorter than about 15 minutes, preferably shorter than 3 minutes, and most preferably shorter than 1 minute.

Other benefits of the solidified layers of the present invention include the presence of a physical barrier that can be formed by the solidified layer itself. In some disease or injury situations, the skin surface is sensitive to the touch of foreign objects or vulnerable to infection if contact by foreign objects. In those situations, the solidified layer can provides physical protection to the skin surface. For instance, local anesthetic agents and other agents such as clonidine may be delivered topically for treating pain related to neuropathy, such as diabetic neuropathic pain. Since many of such subjects feel tremendous pain, even when their skin area is only gently touched, the physical barrier of the solidified layer can prevent or minimize pain caused by accidental contact with objects or others.

Another advantage of the sprayable formulations of the present invention is that they can be applied to a skin surface without the need to touch or rub the skin surface. For example, as noted above, subjects experiencing neuropathic pain often feel pain even when the skin area is only gently touched, such as with an applicator. Sprayable application of the solidifying formulation allows for the application of medicated formulation without the need to touch or rub the skin. For instance, a spray-on solidified formulation of a corticosteroid for treating alopecia can be applied easier than a traditional semi-solid formulation onto a scalp area that has some hair. Another example in which spray application can be beneficial is in damaged or infected skin. Subjects having damaged or infected skin or tissue may not be able to withstand the pain associated with non-spray-on formulations. Additionally, when a formulation requires the subject to physically touch the damaged or infected skin, the risk of a new or increased infection is also increased.

The spray-on formulations can provide other important advantages over currently available “spray formulations” or “semi-solid” formulations. When compared to other spray-on formulations, the solidified layers of the present invention formed from the spray-on solidifying formulations can hold a higher amount of drug for more sustained delivery. When compared to the traditional semi-solid formulations, the sprayable solidified formulations of the present invention can be easier to apply or can be applied without touching the skin surface with an application device such as a spatula. The skin surfaces to which the formulations of the current invention can be applied include but are not limited to skin, mucosal surfaces of the lip, genitals, and anus, nail surface, wound surface, bed sore surface, and diabetes-induced ulcerous skin surface.

The present invention provides a convenient means of applying a solidifying formulation in combination with liquefied propellants to readily form a uniform solidified layer which will remain in intimate contact with the site of application, and provide active drug delivery. This requires applying sufficient quantities of the formulation per unit area of the skin surface so that the solidified layer can contain sufficient amount of the drug. For most drugs the solidified layer needs to be at least 0.01 mm thick, and preferably at least 0.05 mm thick.

These and other advantage can be summarized in the following non-limiting list of benefits. The formulation can be readily sprayed onto a skin surface without the need to touch the surface which could cause pain or infection of the area. The solidified layer comprises a non-volatile solvent system that is flux-enabling for the drug so that the drug can be delivered over sustained period of time at therapeutically effective rates. Further, as the solidified layer remains adhesive to skin and is preferably peelable, easy removal of the solidified layer can occur, and may occur without the aid of a solvent or surfactant. In some embodiments, the adhesion to skin and elasticity of the material is such that the solidified layer will not separate from the skin upon skin stretching at highly stretchable skin areas, such as over joints and muscles. For example, in one embodiment, the solidified layer can be stretched by 5%, or even 10% or greater, in at least one direction without cracking, breaking, and/or separating form a skin surface to which the layer is applied. Still further, the solidified layer can be formulated to advantageously deliver drug and protect sensitive skin areas.

In one embodiment of the invention, the solidified layer may be washed off with a solvent, such as water or ethanol, at the end of the desired drug delivery. Other solvents which could also be used to wash off the solidified formulation include but are not limited to the volatile solvents listed herein. The ability to be removed by washing is particularly advantageous for certain applications. For example, if the solidifying formulation is applied to a skin surface with a lot of hair (e.g. an anti genital herpes solidifying formulation applied on genital skin area with pubic hair), removal by peeling might cause discomfort and therefore be undesirable, and hence washing can be a preferred form of removal in this type of application. In another example, if the solidifying formulation is sprayed onto a palmar surface, such as the palm of the hand or the sole of a foot, the ability for removal by peeling may be secondary consideration to a formulation that will adhere to the skin surface. In these cases, a solidified layer formulated to be easily washed off by water or ethanol can be more desirable. In washing embodiments, the solvent used to wash off the solidified layer may dissolve the layer or make it less adhesive to the skin so that it can be easily removed from the skin. This being stated, it is noted that the solidified layers can be both peelable and washable in some embodiments.

In another embodiment, a solidifying formulation has a viscosity such that it may be dispensed from a container (aerosol or pump-spray) with a metered dose or volumetric control such that a controlled amount of the solidifying formulation is dispensed. The formulation comprises the components as described in the embodiments described above. Controlling the amount of formulation dispensed can avoid under-dosing or overdosing that may lead to undesirable therapeutic effect and/or adverse side effects.

In another embodiment of the present invention, a system having two components includes a solidifying formulation with viscosity such that it may be dispensed from a pressurized container, and a pressurized container. The formulation comprises a solidifying agent, at least one non-volatile solvent, a drug, a propellant, and optionally a volatile solvent system. Once the formulation is sprayed onto a skin surface and after the evaporation of the propellant and the optional volatile solvent(s), the formulation will form a drug-delivering solidified layer on the skin that can be easily removed after use. In this and other embodiments, shortly before use, the aerosol container containing the formulation and the propellant can be shaken to mix the propellant and the rest of the formulation into a temporary suspension which has the appropriate viscosity to be sprayed on a skin surface. Once applied and after the evaporation of the propellant and the optional volatile solvent(s), the formulation forms a drug-delivering solidified layer on the skin that can be easily removed after use. Alternatively, the pressurized container can include a mechanism which causes the propellant to mix with the rest of the formulation to form a mixture that is expelled from the container onto a skin surface. Once applied and after the evaporation of the propellant and the optional volatile solvent(s), the formulation forms a drug-delivering solidified layer on the skin that can be easily removed after use.

In another embodiment, a solidifying formulation has a viscosity such that it may be dispensed from a manual pump-spray container or a conventional pump spray container. The formulation includes a solidifying agent, a non-volatile solvent system, a drug, and optionally a volatile solvent(s). The drug formulation has the appropriate viscosity such that it can easily be expelled from the manual pump-spray container and applied to the skin surface. Once applied the formulation will form a drug-delivering solidified layer on the skin surface that can be easily removed after use.

As a further note, it is a unique feature of the solidified layers of the present invention that they can keep a substantial amount of the non-volatile solvent system, which is optimized for delivering the drug, on the skin surface. This feature can provide unique advantages over existing products. For example, in some semi-solid formulations, upon application to a skin surface, the volatile solvents quickly evaporate and the formulation layer solidifies into a hard lacquer-like layer. The drug molecules are immobilized in the hard lacquer layer and are substantially unavailable for delivery into the skin surface. As a result, it is believed that the delivery of the drug is not sustained over a long period of time. In contrast to this type of formulation, the solidified layers formed using the formulations of the present invention keep the drug molecules quite mobile in the non-volatile solvent system which is in contact with the skin surface, thus ensuring sustained delivery.

Specific examples of applications that can benefit from the systems, formulations, and methods of the present invention are as follows. In one embodiment, a solidified layer can include bupivacaine, lidocaine, tetracaine, and/or ropivacaine, and can be formulated for treating diabetic and post herpetic neuralgia. Alternatively, dibucanine and an alpha-2 agonist such as clonidine can be formulated in a solidifying formulation for treating the same disease. In another embodiment, retinoic acid and benzoyl peroxide can be combined in a solidified layer for treating acne, or alternatively, 1 wt % clindamycin and 5 wt % benzoyl peroxide can alternatively be combined in a solidifying formulation for treating acne. In another embodiment, a retinol solidifying formulation (OTC) can be prepared for treating wrinkles, or a lidocaine solidifying formulation can be prepared for treating back pain. In another embodiment, a zinc oxide solidifying formulation (OTC) can be prepared for treating diaper rash (the physical barrier provided by the solidified layer against irritating urine and feces is believed to be beneficial), or an antihistamine solidified layer can be prepared for treating allergic rashes such as that caused by poison ivy.

Additional applications include delivering drugs for treating certain skin conditions, e.g., dermatitis, psoriasis, eczema, skin cancer, alopecia, wrinkles, viral infections such as cold sore, genital herpes, shingles, etc., particularly those that occur over joints or muscles where a transdermal patch may not be practical. For example, solidifying formulations containing imiquimod can be formulated for treating skin cancer, prematurely aged skin, photo-damaged skin, common and genital warts, and actinic keratosis. Solidifying formulations containing antiviral drugs such as acyclovir, penciclovir, famciclovir, valacyclovir, steroids, and/or behenyl alcohol can be formulated for treating herpes viral infections such as cold sores on the face and genital areas. Solidifying formulations containing non-steroidal anti-inflammatory drugs (NSAIDs), capsaicin, alpha-2 agonists, and/or nerve growth factors can be formulated for treating soft tissue injury and muscle-skeletal pains such as joint and back pain of various causes. As discussed above, patches over these skin areas typically do not have good contact over sustained period of time, especially for a physically active subject, and may cause discomfort. Likewise, traditional semi-solid formulations such as creams, lotions, ointments, etc., may prematurely stop the delivery of a drug due to the evaporation of solvent and/or unintentional removal of the formulation. The solidifying formulations of the present invention address the shortcomings of both of these types of delivery systems.

One embodiment can entail a solidified layer containing a drug from the class of alpha-2 antagonists which is applied topically to treat neuropathic pain. The alpha-2 agonist is gradually released from the formulation to provide pain relief over a sustained period of time. The formulation can become a coherent, soft solid within about 5 minutes and remains adhered to the skin surface for the length of its application, typically hours to tens of hours. The solidified layer is easily removed after the intended application without leaving residual formulation on the skin surface.

Another embodiment involves a solidifying formulation containing capsaicin which is applied topically to treat neuropathic pain. The capsaicin is gradually released from the formulation for treating this pain over a sustained period of time. The formulation can become a coherent, soft solid within about 5 minutes and remains adhered to the skin surface for the length of its application. It can be easily removed any time after drying without leaving residual formulation on the skin surface.

Another embodiment involves a solidifying formulation containing clobetasol propionate which is applied topically to treat hand dermatitis. The clobetasol propionate is gradually released from the formulation for treating dermatitis over a sustained period of time. The formulation can become a coherent, soft solid within about 3 minutes and remains adhered to the skin surface for the length of its application. The physical barrier also protects the compromised skin from potentially harmful substances. It is easily removed any time after drying without leaving residual formulation on the skin surface.

Another embodiment involves a solidifying formulation containing clobetasol propionate which is applied topically to treat alopecia. The clobetasol propionate is gradually released from the formulation for promoting hair growth over a sustained period of time. The formulation can become a coherent, soft solid within about 5 minutes and remains adhered to the skin surface for the length of its application. It is easily removed any time after drying by peeling to showering.

Another embodiment involves sprayable solidifying formulations containing tazorac for treating stretch marks, wrinkles, sebaceous hyperplasia, or seborrheic keratosis.

In another embodiment, solidifying formulations containing glycerol can be made so as to provide a protective barrier for fissuring on finger tips.

Still another embodiment can include a solidifying formulation containing a drug selected from the local anesthetic class, including lidocaine and ropivacaine, or the like, or NSAID class, such as ketoprofen, piroxicam, diclofenac, indomethacin, or the like. These drugs can be applied topically to treat symptoms of back pain, muscle tension, and/or myofascial pain. The local anesthetic and/or NSAID is/are gradually released from the formulation to provide pain relief over a sustained period of time. The formulation can become a coherent, soft solid within about 3 minutes and remain adhered to the skin surface for the length of its application. It is easily removed any time after drying without leaving residual formulation on the skin surface.

A further embodiment involves a solidifying formulation containing at least one alpha-2 agonist drug, at least one tricyclic antidepressant agent, and/or at least one local anesthetic drug which is applied topically to treat neuropathic pain. The drug(s) are gradually released from the formulation to provide pain relief over a sustained period of time. The formulation can become a coherent, soft solid within about 3 minutes and remains adhered to the skin surface for the length of its application. It can be easily removed any time after drying without leaving residual formulation on the skin surface.

A similar embodiment can include a solidifying formulation containing drugs capsaicin and a local anesthetic drug which is applied topically to the skin to provide pain relief. Another embodiment can include a solidifying formulation containing the combination of a local anesthetic and a NSAID. In both of the above embodiments the drugs are gradually released from the formulation to provide pain relief over a sustained period of time. The formulation can become a coherent, soft solid within about 3 minutes and remains adhered to the skin surface for the length of its application. It is easily removed any time after drying without leaving residual formulation on the skin surface.

In another embodiment, solidifying formulations for the delivery of drugs useful for treating the causes or symptoms of diseases involving joints and muscles can also benefit from the systems, formulations, and methods of the present invention. Such diseases that may be applicable include, but not limited to, osteoarthritis (OA), rheumatoid arthritis (RA), joint and skeletal pain of various other causes, myofascial pain, muscular pain, and sports injuries. Drugs or drug classes that can be used for such applications include, but are not limited to, non-steroidal anti-inflammatory drugs (NSAIDs) such as ketoprofen and diclofanac, COX-2 selective NSAIDs and agents, COX-3 selective NSAIDs and agents, local anesthetics such as lidocaine, bupivacaine, ropivacaine, and tetracaine, and steroids such as dexamethasone.

Delivering drugs for the treatment of acne and other skin conditions can also benefit from principles of the present invention, especially when delivering drugs having low skin permeability. Currently, topical retinoids, peroxides, and antibiotics for treating acne are mostly applied as traditional semisolid gels or creams. However, due to the shortcomings as described above, sustained delivery over many hours is unlikely. For example, clindamycin, benzoyl peroxide, and erythromycin may be efficacious only if sufficient quantities are delivered into hair follicles. However, a traditional semisolid formulation, such as the popular acne medicine benzaclin gel, typically loses most of its solvent (water in the case of benzaclin) within a few minutes after the application. This short period of a few minutes likely substantially compromises the sustained delivery of the drug. The formulations of the present invention typically do not have this limitation.

In another embodiment, the delivery of drugs for treating neuropathic pain can also benefit from the methods, systems, and formulations of the present invention. A patch containing a local anesthetic agent, such as Lidoderm™, is widely used for treating neuropathic pain, such as pain caused by post-herpetic neuralgia. Due to the limitations of the patch as discussed above, the solidified layers prepared in accordance with the present invention provide some unique benefits, as well as provide a potentially less expensive alternative to the use of a patch. Possible drugs delivered for such applications include, but are not limited to, local anesthetics such as lidocaine, prilocaine, tetracaine, bupivicaine, etidocaine; and/or other drugs including capsaicin and alpha-2 agonists such as clonidine, dissociative anesthetics such as ketamine, and/or tricyclic antidepressants such as amitriptyline,.

The solidifying formulations of the present invention can be formulated to treat a variety of conditions and disease such as musculoskeletal pain, neuropathic pain, alopecia, skin disease including dermatitis and psoriasis as well as skin restoration (cosmetic skin treatment), and infections including viral, bacterial, and fungal infection. As such the formulations can deliver a wide ranging number and types of drugs and active agents. In one embodiment, the solidifying formulation can be formulated to include acyclovir, econazole, miconazole, terbinafine, lidocaine, bupivacaine, ropivacaine, and tetracaine, amitriptyline, ketanserin, betamethasone dipropionate, triamcinolone acetonide, clindamycin, benzoyl peroxide, tretinoin, Isotretinoin, clobetasol propionate, halobetasol propionate, ketoprofen, piroxicam, diclofenac, indomethacin, imiquimod, salicylic acid, benzoic acid, or combinations thereof In another embodiment, the formulation can include an antifungal drug such as amorolfine, butenafine, naftifine, terbinafine, fluconazole, itraconazole, ketoconazole, posaconazole, ravuconazole, voriconazole, clotrimazole, butoconazole, econazole, miconazole, oxiconazole, sulconazole, terconazole, tioconazole, caspofungin, micafungin, anidulafingin, amphotericin B, AmB, nystatin, pimaricin, griseofulvin, ciclopirox olamine, haloprogin, tolnaftate, and undecylenate, or combinations thereof.

In another embodiment, the formulation can include an antifungal drug such as acyclovir, penciclovir, famciclovir, valacyclovir, behenyl alcohol, trifluridine, idoxuridine, cidofovir, gancyclovir, podofilox, podophyllotoxin, ribavirin, abacavir, delavirdine, didanosine, efavirenz, lamivudine, nevirapine, stavudine, zalcitabine, zidovudine, amprenavir, indinavir, nelfinavir, ritonavir, saquinavir, amantadine, interferon, oseltamivir, ribavirin, rimantadine, zanamivir, or combinations thereof.

When the formulation is intended to provide antibacterial treatment, it can be formulated to include an antibacterial drug such as erythromycin, clindamycin, tetracycline, bacitracin, neomycin, mupirocin, polymyxin B, quinolones such as ciproflaxin, or combinations thereof.

When the formulation is intended to relieve pain, particularly neuropathic pain, the formulation can include a local anesthetic such as lidocaine, bupivacaine, ropivacaine, and/or tetracaine; and/or an alpha-2 agonists such as clonidine. When the formulation is intended to treat pain associated with inflammation, it can be formulated to include an non-steroidal anti-inflammatory drug such as ketoprofen, piroxicam, diclofenac, indomethacin, COX inhibitors general COX inhibitors, COX-2 selective inhibitors, COX-3 selective inhibitors, or combinations thereof.

In another embodiment, the formulation can be formulated to treat skin disorders or blemishes by including active agents such as anti-acne drugs such as clindamycin and benzoyl peroxide, retinol, vitamin A derivatives such as tazarotene and isotretinoin, cyclosporin, anthralin, vitamin D3, cholecalciferol, calcitriol, calcipotriol, tacalcitol, calcipotriene, etc.

In yet another embodiment, the delivery of medication for treating warts and other skin conditions would also benefit from long periods of sustained drug delivery. Examples of anti-wart compounds include but are not limited to:imiquimod, rosiquimod, keratolytic agents: salicylic acid, alpha hydroxy acids, sulfur, rescorcinol, urea, benzoyl peroxide, allantoin, tretinoin, trichloroacetic acid, lactic acid, benzoic acid, or combinations thereof.

A further embodiment involves the use of the solidifying formulations for the delivery of sex steroids including, but not limited to, progestagens consisting of progesterone, norethindrone, norethindroneacetate, desogestrel, drospirenone, ethynodiol diacetate, norelgestromin, norgestimate, levonorgestrel, dl-norgestrel, cyproterone acetate, dydrogesterone, medroxyprogesterone acetate, chlormadinone acetate, megestrol, promegestone, norethisterone, lynestrenol, gestodene, tibolene, androgens consisting of testosterone, methyl testosterone, oxandrolone, androstenedione, dihydrotestosterone, estrogens consisting of estradiol, ethniyl estradiol, estiol, estrone, conjugated estrogens, esterified estrogens, estropipate, or combinations thereof.

Non-sex steroids can also be delivered using the formulations of the present invention. Examples of such steroids include, but are not limited to, betamethasone dipropionate, halobetasol propionate, diflorasone diacetate, triamcinolone acetonide, desoximethasone, fluocinonide, halcinonide, mometasone furoate, betamethasone valerate, fluocinonide, fluticasone propionate, triamcinolone acetonide, fluocinolone acetonide, flurandrenolide, desonide, hydrocortisone butyrate, hydrocortisone valerate, alclometasone dipropionate, flumethasone pivolate, hydrocortisone, hydrocortisone acetate, or combinations thereof.

A further embodiment involves controlled delivery of nicotine for treating nicotine dependence among smokers and persons addicted to nicotine. Formulations of the present invention would be a cost effective way of delivering therapeutic amounts of nicotine transdermally.

Another embodiment involves using the formulation to deliver anti-histamine agents such as diphenhydramine and/or tripelennamine. These agents would reduce itching by blocking the histamine that causes the itch and also provide relief by providing topical analgesia.

Other drugs which can be delivered using the solidifying formulations of the present invention include, but are not limited to, tricyclic anti-depressants such as amitriptyline; anticonvulsants such as carbamazepine and alprazolam; N-methyl-D-aspartate (NMDA) antagonists such as ketamine; 5-HT2A receptor antagonists such as ketanserin; and immune modulators such as tacrolimus and picrolimus. Other drugs that can be delivered using the formulations and methods of the current invention include humectants, emollients, and other skin care compounds.

### EXAMPLES

The following examples illustrate the embodiments of the invention that are presently best known. However, it is to be understood that the following are only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following examples provide further detail in connection with what are presently deemed to be the most practical and preferred embodiments of the invention.

### Example 1

A pressurized container filled with a sprayable solidifying formulation for delivering a drug is prepared which includes a drug (e.g., ketoprofen, testosterone, etc.), a solidifying agents of polyvinyl alcohol (31,000-50,000 Mw) (Amresco) and esters of polyvinylmethylether/maleic anhydride copolymer (80,000-160,000 Mw) (Gantrez ES-425), a non-volatile solvent system of propylene glycol and glycerol, and a volatile solvent system of water and ethanol. The propellant includes at least one of: propane, butane, isobutane, pentane, isopentane, fluro-chloro-hydrocarbons, diethyl ether, dimethyl ether, 1,1 difluoroethane, 1,1,1,2 tetrafluorethane, 1,1,1,2,3,3,3-heptafluoropropane, 1,1,1,3,3,3 hexafluoropropane, vinyl chloride, compressed carbon dioxide, compressed nitrogen, or a combination thereof. By adding a sufficient concentration of the propellant, the container becomes inherently pressurized.

### Example 2

A subject sprays the solidifying formulation prepared similarly as in Example 1 from a pressurized container on an ankle suffering from pain or inflammation caused by an injury or arthritis. The solidifying formulation quickly solidifies into a soft, coherent, and elastic solid layer after the evaporation of the propellant and the volatile solvent(s), and remains in intimate contact with the skin site until removal by the subject. The solidified layer delivers a therapeutically effective amount of ketoprofen across the skin and into the ankle tissues over at least 2 hours, and preferably at least 8 hours, to control pain and inflammation. The non-volatile solvent(s) also keeps the solidified layer soft, coherent, and elastic, as well as provides a flux-enabling solvent in the solidified layer to continuously deliver the ketoprofen through the skin in the absence of water or more volatile solvents and propellants. At the end of the intended application period, the solidified layer can be lifted from the skin due to its good cohesion.

### Example 3

A subject sprays a solidifying formulation containing testosterone prepared similarly as in Example 1 from a pressurized container on his upper arm, shoulders or abdomen area. The solidifying formulation quickly solidifies into a solid layer after the evaporation of the propellant. The solid layer is soft, coherent, elastic, and remains in intimate contact with the skin site until it is removed. The solidified layer delivers therapeutically effective amounts of testosterone across the skin and into the subject's systemic circulation over a period of at least 6 hours. The non-volatile solvent serves as the vehicle for delivering testosterone and also keeps the solidified layer soft, coherent and elastic, as well as provides a flux-enabling solvent in the solidified layer to continuously deliver the testosterone through the skin in the absence of water or more volatile solvents and propellants. At the end of the intended application period, the solidified layer can be removed from the skin due as a single large piece or as several large pieces due to its good cohesion.

### Example 4

A subject sprays solidifying formulation containing tetracaine from a pressurized container onto a skin site of a subject experiencing neuropathic pain. The solidifying formulation includes tetracaine base, a solidifying agent of Plastoid B (neutral copolymer of butyl methacrylate and methyl methacrylate with a 120,000-180,000 Mw range), a non-volatile solvent system of mineral oil and isostearic acid, Gantrez ES-425 (esters of polyvinylmethylether/maleic anhydride copolymer a 80,000-160,000 Mw range for increased adhesion between the solidified layer and the skin), and propellants of dimethyl ether. After a layer of the formulation is sprayed on the skin, the propellant evaporates quickly and the formulation solidifies into a soft, coherent, and flexible solid layer. The solidified layer delivers tetracaine into the skin and controls the neuropathic pain for a sustained period of time.

### Example 5

A subject sprays solidifying formulation containing ropivacaine from an aerosol container onto a skin suffering from neuropathic pain. The solidifying formulation includes ropivacaine base, a solidifying agent Plastoid B (neutral copolymer of butyl methacrylate and methyl methacrylate with a 120,000-180,000 Mw range), a non-volatile solvent system including at least one of tetrahydroxypropyl ethylenediamine, triacetin, span 20, and isostearic acid. The formulation also includes dimethyl ether as a propellant. After a layer of the formulation is sprayed on the skin, the propellant evaporates quickly and the formulation solidifies into a soft, coherent, and flexible solid layer. Ropivacaine is delivered from the solidified layer into the skin over sustained period of time for treating the neuropathic pain.

### Example 6

A subject sprays solidifying formulation containing clobetasol propionate from a pressurized container onto a scalp area where the subject is suffering from alopecia. Although the scalp area is being treated for alopecia, it has some hair, and the spray-on formulation makes the application easier than applying a cream, ointment, or a non-sprayable solidifying formulation. The solidifying formulation includes clobetasol propionate, a solidifying agent of fish gelatin, a non-volatile solvent system of propylene glycol and isostearic acid, fumed silica as a filler (optional), and a fluorocarbon or dimethyl ether as a propellant. After a layer of the formulation is sprayed on the skin, the propellant evaporates quickly and the formulation solidifies into a soft, coherent, and flexible solid layer. A therapeutically effective amount of clobetasol propionate is delivered from this layer into the scalp surface over at least 6 hours for promoting hair growth. After the intended application, the formulation can be washed off in a shower or head wash, as the solidified layer is soluble in water.

### Example 7

A subject sprays a solidifying formulation containing clobetasol propionate from a pressurized container onto a palm skin area where the subject is suffering from hand dermatitis. The formulation is similar to that in Example 6. After a layer of the formulation is sprayed on the skin, the propellant evaporates quickly and the formulation solidifies into a soft, coherent, and flexible solid layer. Clobetasol propionate is delivered in from this solidified layer into the palm skin surface over at least 2 hours for suppressing the hand dermatitis. The solidified layer is adhesive to the skin and also acts as a physical barrier to protect the skin from external substances that can cause or aggravate the dermatitis.

### Example 8

A subject sprays a solidifying formulation containing an antibiotic agent from a pressurized container onto a skin area of bed sore or diabetes-induced ulcer. The formulation includes an antibiotic, solidifying agent of polyvinyl alcohol, a non-volatile solvent system of glycerol, and a propellant of 1,1,1,2,3,3,3-heptafluoropropane (propellant). After a layer of the formulation is sprayed on the skin, the propellant evaporates quickly and the formulation solidifies into a soft, coherent, and flexible solid layer. The antibiotic agent is delivered at therapeutically effective rates from this layer into the compromised skin surface over at least 2 hours for treating or preventing infection. The solidified layer is adhesive to the skin surface and also acts as a physical barrier to protect the compromised skin area from external pathogens and touch by external objects that can cause pain.

### Example 9

A subject sprays solidifying formulation containing clobetasol propionate from an aerosol container onto a skin area where the subject is suffering from psoriasis. The formulation includes the clobetasol propionate, a solidifying agent of polyvinyl alcohol, a non-volatile solvent system of glycerol, propylene glycol, and oleic acid, Gantrez ES-425 (esters of polyvinylmethylether/maleic anhydride copolymer with a 80,000-160,000 Mw range for increased adhesion between the solidified layer and the skin), and a hydrofluorocarbon as a propellant. After a layer of the formulation is sprayed on the skin, the propellant evaporates quickly and the formulation solidifies into a soft, coherent, and flexible solid layer. Clobetasol propionate is delivered from this layer into the psoriatic skin surface over at least 2 hours, and preferably over at least 6 hours, for suppressing the psoriasis.

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. A spray-on formulation for dermal drug delivery, comprising:
a drug;
a non-volatile solvent system comprising at least one non-volatile solvent;
a solidifying agent; and
a propellant,
wherein the formulation has an initial viscosity suitable to be expelled out of a pressurized container or manual pump container and applied onto a skin surface as a layer, and wherein the formulation is also capable of forming a solidified layer on the skin surface after evaporation of at least a portion of the propellant.

2. A formulation as in claim 1, wherein the non-volatile solvent system is capable of delivering therapeutically effective amount of the drug into or across the skin surface.

3. A formulation as in claim 1, wherein the solidified layer is capable of delivering therapeutically effective amount of the drug into or across the skin surface.

4. A formulation as in claim 1, wherein the formulation is a homogenous solution.

5. A formulation as in claim 1, wherein the formulation is a suspension.

6. A formulation as in claim 1, wherein the propellant exists in a separate phase from other components of the formulation when the formulation is in the pressurized container, and wherein the propellant and the other components are mixed prior to expulsion from the pressurized container.

7. A formulation as in claim 1, wherein the propellant is maintained separately from other components of the formulation when the formulation is in the pressurized container, and wherein the propellant and the other components are mixed shortly before application.

8. A formulation as in claim 1, wherein the formulation further comprises a volatile solvent having a boiling point which is higher than 25° C.

9. A formulation as in claim 1, wherein the formulation further comprises a volatile solvent which improves compatibility of components in the formulation.

10. A formulation as in claim 1, wherein the formulation further comprises a volatile solvent selected from the group consisting of ethanol, isopropyl alcohol, and combinations thereof.

11. A formulation as in claim 1, wherein the formulation further includes a volatile solvent which adjusts the viscosity of the formulation.

12. A formulation as in claim 1, wherein the non-volatile solvent system is a plasticizer for the solidifying agent.

13. A formulation as in claim 1, wherein the solidifying agent is at least ten percent of the total weight of the formulation.

14. A formulation as in claim 1, wherein the solidifying agent is at least twenty percent of the total weight of the formulation.

15. A formulation as in claim 1, wherein the non-volatile solvent system is at least ten percent of the total weight of the formulation.

16. The formulation of claim 1, wherein the non-volatile solvent system is at least twenty percent of the total weight of the formulation.

17. A formulation as in claim 1, wherein the drug includes an active agent for treating neuropathic pain.

18. A formulation as in claim 1, wherein the drug includes a local anesthetic.

18. A formulation as in claim 1, wherein the drug includes ropivacaine.

20. A formulation as in claim 1, wherein the drug includes tetracaine.

21. A formulation as in claim 1, wherein the drug includes lidocaine.

22. A formulation as in claim 1, wherein the drug includes amytriptylene.

23. A formulation as in claim 1, wherein the drug includes a male hormone.

24. A formulation as in claim 1, wherein the drug includes testosterone.

25. A formulation as in claim 1, wherein the drug includes a corticosteroid.

26. A formulation as in claim 1, wherein the drug includes clobetasol.

27. A formulation as in claim 1, wherein the drug includes clobetasol propionate.

28. A formulation as in claim 1, wherein the drug includes an anti-inflammatory agent.

29. A formulation as in claim 1, wherein the drug includes ketoprofen.

30. A formulation as in claim 1, wherein the drug includes an antibiotic agent.

31. A formulation as in claim 1, wherein the drug includes an anti fungal agent.

32. A formulation as in claim 1, wherein the drug includes is an antiviral agent.

33. A formulation as in claim 1, wherein the drug includes an immune modulating agent.

34. A formulation as in claim 1, wherein the drug includes imiquimod.

35. A formulation as in claim 1, wherein the drug includes an anti-infection agent.

36. A formulation as in claim 1, wherein the drug includes at least one member selected from the group consisting of acyclovir, econazole, miconazole, terbinafine, lidocaine, bupivacaine, ropivacaine, and tetracaine, amitriptyline, ketanserin, betamethasone dipropionate, triamcinolone acetonide, clindamycin, benzoyl peroxide, tretinoin, isotretinoin, clobetasol propionate, halobetasol propionate, ketoprofen, piroxicam, diclofenac, indomethacin, imiquimod, salicylic acid, benzoic acid, and combinations thereof.

37. A formulation as in claim 1, wherein the drug includes at least one member selected from the group consisting of amorolfine, butenafine, naftifine, terbinafine, fluconazole, itraconazole, ketoconazole, posaconazole, ravuconazole, voriconazole, clotrimazole, butoconazole, econazole, miconazole, oxiconazole, sulconazole, terconazole, tioconazole, caspofungin, micafungin, anidulafingin, amphotericin B, AmB, nystatin, pimaricin, griseofulvin, ciclopirox olamine, haloprogin, tolnaftate, undecylenate, penciclovir, famciclovir, valacyclovir, behenyl alcohol, trifluridine, idoxuridine, cidofovir, gancyclovir, podofilox, podophyllotoxin, ribavirin, abacavir, delavirdine, didanosine, efavirenz, lamivudine, nevirapine, stavudine, zalcitabine, zidovudine, amprenavir, indinavir, nelfinavir, ritonavir, saquinavir, amantadine, interferon, oseltamivir, ribavirin, rimantadine, zanamivir, erythromycin, clindamycin, tetracycline, bacitracin, neomycin, mupirocin, polymyxin B, quinolones, ciproflaxin, bupivacaine, alpha-2 agonists, clonidine, amitriptyline, carbamazepine, alprazolam, ketamine, ketanserin, betamethasone dipropionate, halobetasol propionate, diflorasone diacetate, triamcinolone acetonide, desoximethasone, fluocinonide, halcinonide, mometasone furoate, betamethasone valerate, fluocinonide, fluticasone propionate, triamcinolone acetonide, fluocinolone acetonide, flurandrenolide, desonide, hydrocortisone butyrate, hydrocortisone valerate, alclometasone dipropionate, flumethasone pivolate, hydrocortisone, hydrocortisone acetate, tacrolimus, picrolimus, tazarotene, isotretinoin, cyclosporin, anthralin, vitamin D3, cholecalciferol, calcitriol, calcipotriol, tacalcitol, calcipotriene, piroxicam, diclofenac, indomethacin, imiquimod, rosiquimod, salicylic acid, alpha hydroxy acids, sulfur, rescorcinol, urea, benzoyl peroxide, allantoin, tretinoin, trichloroacetic acid, lactic acid, benzoic acid, progesterone, norethindrone, norethindroneacetate, desogestrel, drospirenone, ethynodiol diacetate, norelgestromin, norgestimate, levonorgestrel, dl-norgestrel, cyproterone acetate, dydrogesterone, medroxyprogesterone acetate, chlormadinone acetate, megestrol, promegestone, norethisterone, lynestrenol, gestodene, tibolene, testosterone, methyl testosterone, oxandrolone, androstenedione, dihydrotestosterone, estradiol, ethniyl estradiol, estiol, estrone, conjugated estrogens, esterified estrogens, estropipate, and combinations thereof.

38. A formulation as in claim 1, wherein the solidifying agent includes at least one member selected from the group consisting of polyvinyl alcohol, esters of polyvinylmethylether/maleic anhydride copolymer, neutral copolymers of butyl methacrylate and methyl methacrylate, dimethylaminoethyl methacrylate-butyl methacrylate-methyl methacrylate copolymers, ethyl acrylate-methyl methacrylate-trimethylammonioethyl methacrylate chloride copolymers, prolamine (Zein), pregelatinized starch, ethyl cellulose, fish gelatin, gelatin, acrylates/octylacrylamide copolymers, and combinations thereof.

39. A formulation as in claim 1, wherein the solidifying agent includes at least one member selected from the group consisting of ethyl cellulose, hydroxy ethyl cellulose, hydroxy methyl cellulose, hydroxy propyl cellulose, hydroxypropyl methyl cellulose, carboxymethyl cellulose, methyl cellulose, polyether amides, corn starch, pregelatinized corn starch, polyether amides, shellac, polyvinyl pyrrolidone, polyisobutylene rubber, polyvinyl acetate phthalate, and combinations thereof.

40. A formulation as in claim 1, wherein the solidifying agent includes at least one member selected from the group consisting of ammonia methacrylate, carrageenan, cellulose acetate phthalate aqueous, carboxy polymethylene, cellulose acetate (microcrystalline), cellulose polymers, divinyl benzene styrene, ethylene vinyl acetate, silicone, guar gum, guar rosin, gluten, casein, calcium caseinate, ammonium caseinate, sodium caseinate, potassium caseinate, methyl acrylate, microcrystalline wax, polyvinyl acetate, PVP ethyl cellulose, acrylate, PEG/PVP, xantham gum, trimethyl siloxysilicate, maleic acid/anhydride colymers, polacrilin, poloxamer, polyethylene oxide, poly glactic acid/poly-l-lactic acid, turpene resin, locust bean gum, acrylic copolymers, polyurethane dispersions, dextrin, polyvinyl alcohol-polyethylene glycol co-polymers, methyacrylic acid-ethyl acrylate copolymers, methacrylic acid and methacrylate based polymers such as poly(methacrylic acid), and combinations thereof.

41. A formulation as in claim 1, wherein the non-volatile solvent system includes at least one member selected from the group consisting of glycerol, propylene glycol, isostearic acid, oleic acid, propylene glycol, trolamine, tromethamine, triacetin, sorbitan monolaurate, sorbitan monooleate, sorbitan monopalmitate, butanol, and combinations thereof.

42. A formulation as in claim 1, wherein the non-volatile solvent system comprises one or more solvents selected from the group consisting of benzoic acid, butyl alcohol, dibutyl sebecate, diglycerides, dipropylene glycol, eugenol, fatty acids, isopropyl myristate, mineral oil, oleyl alcohol, vitamin E, triglycerides, sorbitan fatty acid surfactants, triethyl citrate, and combinations thereof.

43. A formulation as in claim 1, wherein the non-volatile solvent system includes at least one member selected from the group consisting of 1,2,6-hexanetriol, alkyltriols, alkyldiols, acetyl monoglycerides, tocopherol, alkyl dioxolanes, p-propenylanisole, anise oil, apricot oil, dimethyl isosorbide, alkyl glucoside, benzyl alcohol, bees wax, benzyl benzoate, butylene glycol, caprylic/capric triglyceride, caramel, cassia oil, castor oil, cinnamaldehyde, cinnamon oil, clove oil, coconut oil, cocoa butter, cocoglycerides, coriander oil, corn oil, coriander oil, corn syrup, cottonseed oil, cresol, cyclomethicone, diacetin, diacetylated monoglycerides, diethanolamine, dietthylene glycol monoethyl ether, diglycerides, ethylene glycol, eucalyptus oil, fat, fatty alcohols, flavors, liquid sugars, ginger extract, glycerin, high fructose corn syrup, hydrogenated castor oil, IP palmitate, lemon oil, lime oil, limonene, milk, monoacetin, monoglycerides, nutmeg oil, octyidodecanol, olive alcohol, orange oil, palm oil, peanut oil, PEG vegetable oil, peppermint oil, petrolatum, phenol, pine needle oil, polypropylene glycol, sesame oil, spearmint oil, soybean oil, vegetable oil, vegetable shortening, vinyl acetate, wax, 2-(2-(octadecyloxy)ethoxy)ethanol, benzyl benzoate, butylated hydroxyanisole, candelilla wax, carnauba wax, ceteareth-20, cetyl alcohol, polyglyceryl, dipolyhydroxy stearate, PEG-7 hydrogenated castor oil, diethyl phthalate, diethyl sebacate, dimethicone, dimethyl phthalate, PEG fatty acid esters, PEG-stearate, PEG-oleate, PEG laurate, PEG fatty acid diesters, PEG-dioleate, PEG-distearate, PEG-castor oil, glyceryl behenate, PEG glycerol fatty acid esters, PEG glyceryl laurate, PEG glyceryl stearate, PEG glyceryl oleate, hexylene glycerol, lanolin, lauric diethanolamide, lauryl lactate, lauryl sulfate, medronic acid, methacrylic acid, multisterol extract, myristyl alcohol, neutral oil, PEG-octyl phenyl ether, PEG-alkyl ethers, PEG-cetyl ether, PEG-stearyl ether, PEG-sorbitan fatty acid esters, PEG-sorbitan diisosterate, PEG-sorbitan monostearate, propylene glycol fatty acid esters, propylene glycol stearate, propylene glycol, caprylate/caprate, sodium pyrrolidone carboxylate, sorbitol, squalene, stear-o-wet, triglycerides, alkyl aryl polyether alcohols, polyoxyethylene derivatives of sorbitan-ethers, saturated polyglycolyzed C8-C10 glycerides, N-methyl pyrrolidone, honey, polyoxyethylated glycerides, dimethyl sulfoxide, azone and related compounds, dimethylformamide, N-methyl formamaide, fatty acid esters, fatty alcohol ethers, alkyl-amides (N,N-dimethylalkylamides), N-methyl pyrrolidone related compounds, ethyl oleate, polyglycerized fatty acids, glycerol monooleate, glyceryl monomyristate, glycerol esters of fatty acids, silk amino acids, PPG-3 benzyl ether myristate, Di-PPG2 myreth 10-adipate, honeyquat, sodium pyroglutamic acid, abyssinica oil, dimethicone, macadamia nut oil, limnanthes alba seed oil, cetearyl alcohol, PEG-50 shea butter, shea butter, aloe vera juice, phenyl trimethicone, hydrolyzed wheat protein, and combinations thereof.

44. A formulation as in claim 1, wherein the propellant includes dimethyl ether.

45. A formulation as in claim 1, wherein the propellant includes a hydrofluorocarbon.

46. A formulation as in claim 1, wherein the propellant includes a hydrochlorofluorocarbon.

47. A formulation as in claim 1, wherein the propellant includes at least one member selected from the group consisting of propane, butane, isobutane, pentane, isopentane, fluro-chloro-hydrocarbons, diethyl ether, dimethyl ether, 1,1 difluoroethane, 1,1,1,2 tetrafluorethane, 1,1,1,2,3,3,3-heptafluoropropane, 1,1,1,3,3,3 hexafluoropropane, vinyl chloride, compressed carbon dioxide, compressed nitrogen, and combinations thereof.

48. A formulation as in claim 1, wherein the formulation further comprises an agent capable of increasing the adhesion between the solidified layer and the skin surface.

49. A formulation as in claim 1, wherein the skin surface is skin.

50. A formulation as in claim 1, wherein the skin surface is a mucosal surface.

51. A formulation as in claim 1, wherein the skin surface is a nail surface.

52. A formulation as in claim 1, wherein the skin surface is wound surface.

53. A formulation as in claim 1, wherein the skin surface is bed sore surface.

54. A formulation as in claim 1, wherein the skin surface is a diabetes-induced ulcerous skin surface.

55. A formulation as in claim 1, wherein the weight ratio of the non-volatile solvent system to the solidifying agent is from about 0.5:1 to 2:1.

56. A formulation as in claim 1, wherein the solidified layer provides sustained release of the drug for at least two hours.

57. A formulation as in claim 1, wherein the solidified layer provides sustained release of the drug for at least 6 hours.

58. A formulation as in claim 1, wherein the solidified layer is a soft, coherent solid that is removable from the skin after use by peeling as a single piece or a few large pieces relative to the application area.

59. A formulation as in claim 1, wherein the formulation is contained in the pressurized container.

60. A formulation as in claim 1, wherein the formulation is contained in the manual pump container.

61. A formulation as in claim 1, wherein the solidified layer is formulated to deliver the drug transdermally.

62. A method for dermal drug delivery, comprising
a) spraying onto a skin surface an adhesive, solidifying formulation, the formulation comprising:
i) a drug,
ii) a non-volatile solvent system comprising at least one non-volatile solvent, the non-volatile solvent system being flux-enabling for the drug,
iii) a solidifying agent, and
iv) a propellant;
wherein the formulation has an initial viscosity suitable to be expelled out of a pressurized container and applied onto a skin surface as a layer;
b) solidifying the formulation to form a solidified layer on the skin surface by at least partial evaporation of the propellant; and
c) dermally delivering the drug from the solidified layer to the skin surface at a therapeutically effective rate over a sustained period of time.

63. A method as in claim 62, wherein the solidified layer is kept on the skin surface for at least about 2 hours.

64. A method as in claim 62, wherein the solidified layer is kept on the skin surface for at least about 6 hours.

65. A method as in claim 62, wherein the step of spraying is by spraying the formulation from a pressurized container onto the skin surface.

66. A method as in claim 62, wherein the spraying is by spraying the formulation from a container using a manual pump onto the skin surface.

67. A method as in claim 62, wherein the solidified layer is at least 0.01 mm thick.

68. A method as in claim 62, wherein the solidified layer is at least 0.05 mm thick.

69. A method as in claim 62, wherein the skin surface is a mucosal surface.

70. A method as in claim 62, wherein the skin surface is a nail surface.

71. A method as in claim 62, wherein the skin surface is a wounded skin surface.

72. A method as in claim 62, wherein the skin surface is a skin surface afflicted with a bed sore.

73. A method as in claim 62, wherein the skin surface is a diabetes-induced ulcerous skin surface.

74. A method as in claim 62, wherein the step of delivering the drug occurs primarily in the substantial absence of the propellant, water, and any solvents more volatile than water.
